(19) United States
(12) Patent Application Publication
(10) Pub. No.: US 2023/0296576 A1
(43) Pub. Date: Sep. 21, 2023

(54) CHEMICAL SENSOR DEVICE AND METHOD FOR DETECTING TARGET SUBSTANCE

(71) Applicant: KABUSHIKI KAISHA TOSHIBA, TOKYO (JP)

(72) Inventors: Yoshiaki SUGIZAKI, Fujisawa Kanagawa (JP); Hiroko MIKI, Kawasaki Kanagawa (JP)

(73) Assignee: KABUSHIKI KAISHA TOSHIBA, TOKYO (JP)

(21) Appl. No.: 17/939,603

(22) Filed: Sep. 7, 2022

(30) Foreign Application Priority Data
Mar. 18, 2022 (JP) 2022-044478

(51) Int. Cl. G01N 33/00; G01N 1/40; G01N 27/40

(52) U.S. Cl. CPC G01N 33/0031; G01N 1/4005; G01N 27/40; G01N 33/0036; G01N 33/0062

(57) ABSTRACT

Embodiments provide a chemical sensor device that is a chemical sensor using a nucleic acid compound and a specific blocking agent and can detect a target substance with high sensitivity, and a method for detecting a target substance.

A chemical sensor device of an embodiment includes a sensitive film, a nucleic add compound that is immobilized on the sensitive film, and a phosphoric acid derivative that is immobilized on a surface of the sensitive film on which the nucleic add compound is immobilized.

### CROSS REFERENCE TO RELATED APPLICATIONS

This application is based upon and claims the benefit of priority from the prior Japanese Patent Application No. 2022-44478, filed on Mar. 18, 2022, the entire contents of which are incorporated herein by reference.

### FIELD

Embodiments according to the present invention relate to a chemical sensor device and a method for detecting a target substance.

### BACKGROUND

A graphene film or a carbon nanotube exhibits a large change in electrical characteristics (high sensitivity) with respect to binding, adsorption, or proximity of a substance on a surface thereof. By utilizing this property, a highly sensitive sensor has been proposed in which a molecule (hereinafter, referred to as “probe molecule”) associated with a specific chemical substance, a nucleic acid, a virus, or a microorganism (hereinafter, referred to as “target substance”) is disposed on a surface of a graphene film or a carbon nanotube and association between the probe molecule and the target substance is detected.

Many probe molecules have been proposed utilizing the ability to selectively recognize a specific substance unique to an organism, and enzymes having the ability to selectively promote a specific chemical reaction, antibodies that recognize and capture foreign substances, nonself, and pathogens in the immune system of an organism, aptamers associated with a specific substance by a nucleic acid base sequence, peptide aptamers associated with a specific substance by an amino acid sequence, and the like are used.

As the target substance, viruses and microorganisms that cause infectious illness and diseases, nucleic acids, endocrine substances, and blood cells that are indicators of disease states and health conditions, low molecular weight compounds that are odor components or volatile organic chemicals, and the like have been widely proposed.

### BRIEF DESCRIPTION OF THE DRAWINGS

FIG. 1 is a cross-sectional view showing an example of a chemical sensor device of an embodiment.

FIG. 2 is a schematic view showing an example of a nucleic acid used in the chemical sensor device of the embodiment.

FIG. 3 is a schematic view showing a state when the chemical sensor device of the embodiment is used.

FIG. 4 is a graph showing a time-dependent change in drain current change rate at the time of manufacturing a chemical sensor device of Example 1.

FIG. 5 is a graph showing a time-dependent change in drain current change rate at the time of manufacturing a chemical sensor device of Comparative Example 1.

FIG. 6 is a graph showing a time-dependent change in drain current change rate at the time of manufacturing a chemical sensor device of Comparative Example 2.

FIG. 7 is a graph showing a time-dependent change in drain current change rate at the time of manufacturing a chemical sensor device of Comparative Example 3.

FIG. 8 is a graph showing a time-dependent change in drain current change rate at the time of manufacturing a chemical sensor device of Comparative Example 4.

FIG. 9 is a graph showing a time-dependent change in drain current change rate at the time of manufacturing a chemical sensor device of Comparative Example 5,

### DETAILED DESCRIPTION

Embodiments according to the present invention provide a chemical sensor device that is a chemical sensor using a nucleic acid compound and a specific blocking agent and can detect a target substance with high sensitivity, and a method for detecting a target substance.

A chemical sensor device according to an embodiment includes a sensitive film, a nucleic acid compound that is immobilized on said sensitive film, and a phosphoric acid derivative that is immobilized on a surface of said sensitive film on which said nucleic acid compound is immobilized.

A method for detecting a target substance according to another embodiment includes, in the stated order, exposing a sensitive film to a first solution containing a phosphoric acid derivative, exposing said sensitive film to a second solution containing a nucleic acid compound, and exposing said sensitive film to a specimen liquid capable of containing a target substance.

Hereinafter, various embodiments will be described with reference to the drawings. Each drawing is a schematic view for explaining the embodiments and facilitating understandings thereof, and shapes, dimensions, ratios and the like in the drawing may be different from the actual shapes, dimensions, ratios and the like, but their designs can be appropriately changed by referring to the following descriptions and publicly known techniques.

In a chemical sensor using a nucleic acid (for example, DNA), a target substance to be detected is captured by the nucleic acid to change the conformation of the nucleic acid, and a detection mechanism for reading the change is used, Since the method using this mechanism does not detect the target substance itself, it is particularly effective when an uncharged low molecular weight compound is to be detected.

In order to express such a mechanism, it is preferable to immobilize the nucleic acid on a surface of a sensitive film at a specific site and capture the target substance at another site of the nucleic acid (specific adsorption).

However, in a chemical sensor using graphene or the like for a sensitive film, when the nucleic acid is immobilized on a surface of the sensitive film, even a site to be specifically adsorbed is adsorbed to the sensitive film (non-specific adsorption), and the above mechanism may not be expressed.

As a result of various studies on a chemical sensor device using a nucleic acid, the present inventors have found that the adsorption of the nucleic acid to the sensitive film at a site where specific adsorption is to be performed can be suppressed by inhibiting an interaction between a phosphoric acid ester moiety, which is the main skeleton of the nucleic acid, and the sensitive film, Specifically, the present inventors have found that it is effective to expose the surface of the sensitive film to high concentration phosphoric add in advance and occupy an adsorption site on the surface of the sensitive film with phosphoric acid in order to suppress adsorption of the phosphoric acid ester moiety of the nucleic acid to the sensitive film.

However, the present inventors have also investigated that high concentration phosphoric acid interferes with a signal of a negative charge of the phosphoric acid ester moiety, which is the main skeleton of the nucleic acid, and as a result, a target substance may not be detected.

Based on the results of the investigation, the present inventors have found that it is effective to use a phosphoric acid derivative as a blocking agent instead of phosphoric acid.

As described above, according to the embodiment, even in the chemical sensor device using a nucleic acid, a target substance can be detected with high sensitivity.

Hereinafter, a chemical sensor device of an embodiment will be described.

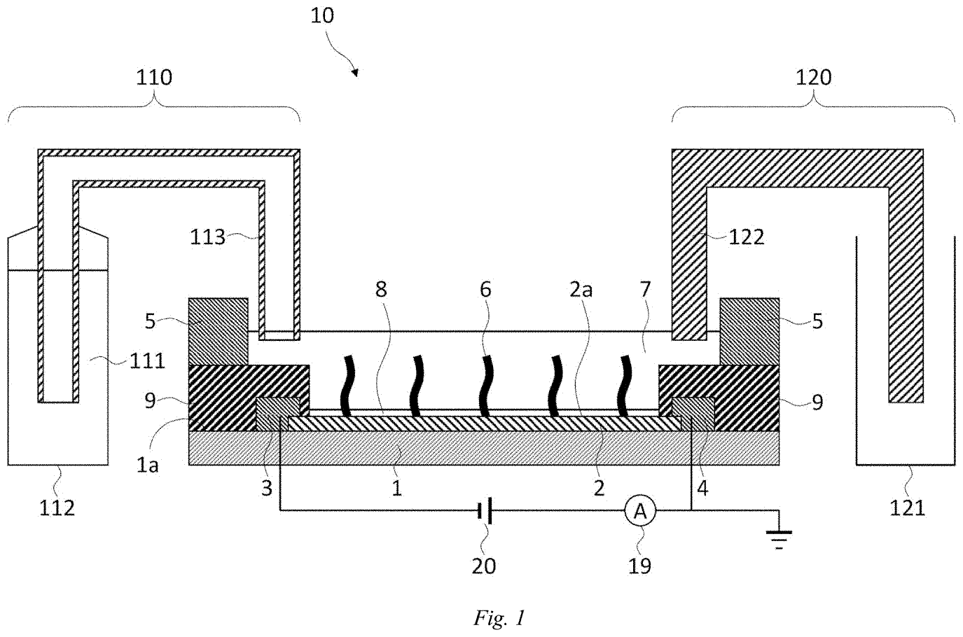

FIG. 1 is a cross-sectional view showing an example of a chemical sensor device of an embodiment.

A chemical sensor device 10 includes a substrate 1. A sensitive film 2, a source electrode 4 connected to one end of the sensitive film 2, and a drain electrode 3 connected to the other end of the sensitive film 2 are provided on a surface 1a of the substrate 1. A protective insulating film 9 is further formed so as to cover each of the source electrode 4 and the drain electrode 3, and the protective insulating film 9 is opened so as to expose at least a part of the sensitive film 2. A wall portion 5 is erected on the protective insulating film 9 or the substrate 1, and the wall portion 5 covers the outer peripheral surface of the sensitive film 2 so as to expose a portion including the sensitive film 2 in plan view. The wall portion 5 has a function of holding a liquid on a surface 2a of the sensitive film 2. A nucleic acid compound 6 as a receptor is immobilized on the surface 2a of the sensitive film 2. The surface 2a of the sensitive film 2 is a surface on which the nucleic acid compound 6 is immobilized, and a portion of the surface 2a on which the nucleic acid compound 6 is not immobilized is covered with a blocking agent 8. When a target substance 21 is detected by the chemical sensor device 10, a liquid film 7 is disposed on the surface 2a of the sensitive film 2 so as to cover the nucleic acid compound 6 and the blocking agent 8, In the embodiment, “covering” means covering at least a part of the surface. The liquid film 7 includes a specimen liquid 111 capable of containing the target substance 21, The chemical sensor device 10 can include a liquid supply mechanism 110 that supplies a specimen liquid to the surface 2a of the sensitive film 2 and a liquid discharge mechanism 120 that discharges the specimen liquid on the surface 2a of the sensitive film 2.

The liquid supply mechanism 110 can supply a liquid to the liquid film 7. As shown in FIG. 1, the liquid supply mechanism 110 includes a first container (bottle) 112 which is a supply source of the specimen liquid 111 and is installed to be separated from the wall portion 5. One end of a capillary 113 is inserted into the specimen liquid 111 accommodated in the first container 112. The other end of the capillary 113 is disposed in contact with the liquid film 7. The capillary 113 feeds the specimen liquid 111 in the first container 112 to the liquid film 7. The capillary 113 is formed, for example, from a material such as glass, and the inner surface of the capillary 113 preferably has hydrophilicity.

The liquid supply mechanism 110 can supply the specimen liquid 111 in the first container 112 to the liquid film 7 through the capillary 113. This supply can utilize capillary phenomenon. In such liquid supply, the surface 2a of the sensitive film 2 preferably has hydrophilicity, and the liquid supplied to the liquid film 7 can quickly permeate and diffuse into the entire surface 2a of the sensitive film 2 utilizing capillary phenomenon. The liquid supply mechanism 110 does not necessarily include a capillary, and can be realized by an arbitrary configuration capable of conveying a liquid, such as a dropping device or a pump.

The liquid discharge mechanism 120 can discharge the liquid in the liquid film 7. As shown in FIG. 1, the liquid discharge mechanism 120 includes a second container 121 which collects the discharged liquid and is installed to be separated from the wall portion 5. One end of an absorbent material 122 is inserted into the second container 121. The other end of the absorbent material 122 is disposed in contact with the liquid film 7. The absorbent material 122 absorbs the liquid in the liquid film 7 and feeds the liquid to the second container 121, The absorbent material 122 is formed, for example, from a hygroscopic material or water-absorbent material selected from sodium polyacrylate, polyethylene, polystyrene, and the like. The absorbent material 122 may have a capillary structure in the same manner as the capillary 113 in the liquid supply mechanism 110, or may have a configuration in which a liquid exceeding the capacity of the wall portion 5 overflows.

In the chemical sensor device 10, the liquid is supplied from one end of the liquid film 7 by the liquid supply mechanism 110, and the liquid in the liquid film 7 is discharged from the other end of the liquid film 7 by the liquid discharge mechanism 120, so that a flow of the liquid from one end to the other end of the liquid film 7 is generated, and a state where the nucleic acid compound 6 is wetted by the liquid film 7 can be maintained. The liquid supply mechanism 110 and the liquid discharge mechanism 120 can maintain the liquid film 7 to a thickness of, for example, 0.5 μm or more and 10.0 μm or less.

Hereinafter, each configuration will be described in detail.

The substrate 1 has, for example, a rectangular plate shape. The substrate 1 is formed, for example, from silicon, silicon oxide, glass, ceramics, a polymer material, a metal, or the like. The size of the substrate 1 is not limited, and is, for example, 1 to 10 mm×1 to 10 mm×0.1 to 1 mm (width×length×thickness).

The protective insulating film 9 is formed, for example, from an electrically insulating material selected from silicon oxide, silicon nitride, aluminum oxide, a fluororesin, a polymer material, a self-assembled film of organic molecules, and the like. Alternatively, when polarizable electrodes are used for the source electrode 4 and the drain electrode 3, the protective insulating film 9 may not be provided. The polarizable electrode is an electrode formed from a material having insulation property with the liquid film 7 and a potential window, and is made of, for example, gold, platinum, carbon, or the like. The substrate 1 may further include a conductor layer functioning as a gate electrode. The gate electrode is preferably a non-polarizable electrode, and for example, a silver/silver chloride electrode or the like can be used. Alternatively, the gate electrode may not be provided on the substrate 1, and may be brought into contact with the liquid film 7 as a separate part.

The sensitive film 2 is a film whose physical properties change when the structure of a substance bonded to the surface thereof, a state of charge, or the like changes. The sensitive film 2 is formed, for example, from a substance whose electric resistance changes. The sensitive film 2 is preferably a single layer graphene film having a thickness corresponding to one carbon atom. The graphene film may be provided in a plurality of layers. The size of the sensitive film 2 is not limited, and can be set, for example, to 0.1 to 500 μm×0.1 to 500 μm (width×length). Practically, when the size of the sensitive film 2 is 10 to 100 μm×10 to 100 μm, the sensitive film 2 is easy to produce.

The sensitive film 2 may be formed, for example, from a material containing graphene, carbon nanotubes, molybdenum disulfide (MoS₂), tungsten diselenide (WSe₂), or the like.

The source electrode 4 and the drain electrode 3 are formed, for example, from a metal such as gold (Au), palladium (Pd), platinum (Pt), nickel (Ni), titanium (Ti), chromium (Cr), or aluminum (Al), or a conductive material such as zinc oxide (ZnO), indium tin oxide (ITO), IGZO (oxide semiconductor of indium, gallium, and zinc), or a conductive polymer.

The source electrode 4 and the drain electrode 3 are electrically connected to a power supply 20. The source electrode 4 and the drain electrode 3 are configured such that, for example, when a voltage (source-drain voltage (V_{sd})) is applied from the power supply 20, a current (source-drain current (I_{sd})) flows from the source electrode 4 to the drain electrode 3 through the sensitive film 2. At this time, when the sensitive film 2 is a graphene film, the sensitive film 2 functions as a channel with respect to the source electrode 4 and the drain electrode 3. The magnitude of the source-drain current can be measured by an ammeter 19, By applying a predetermined gate voltage to the gate electrode, the magnitude of the source-drain current corresponding to the gate voltage can be measured. That is, a field effect transistor signal (FET signal) can be detected by the source electrode 4 and the drain electrode 3 using the sensitive film 2 and the ammeter 19.

The wall portion 5 is formed, for example, from an electrically insulating material, Examples of the insulating material for the wall portion 5 include polymer substances such as an acrylic resin, polyimide, polybenzoxazole, an epoxy resin, a phenol resin, polydimethylsiloxane, and a fluororesin, and inorganic insulating films such as silicon oxide, silicon nitride, and aluminum oxide.

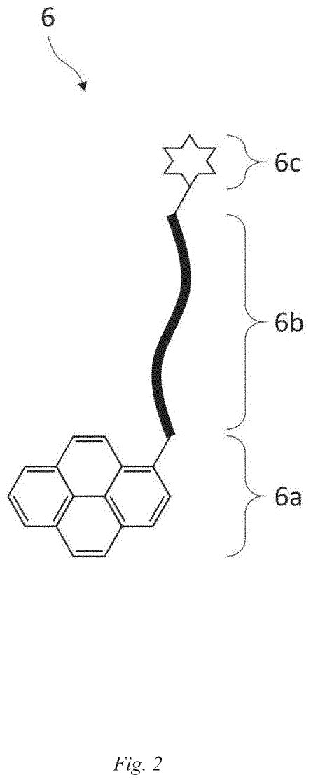

The nucleic acid compound 6 has, for example, a site suitable for binding to the sensitive film 2 (hereinafter, referred to as “immobilized site”) 6a and a site suitable for binding to the target substance 21 (hereinafter, referred to as “detection site”) 6b, as shown in FIG. 2. In the embodiment, for example, the nucleic acid compound 6 in which one end is the immobilized site 6a and the other end is the detection site 6b is used, Since the immobilized site 6a of the nucleic acid compound 6 binds to an adsorption site on the surface 2a of the sensitive film 2, the nucleic acid compound 6 is immobilized on the surface 2a of the sensitive film 2. When the detection site 6b of the nucleic acid compound 6 comes into contact with the liquid film 7, the detection site 6b easily captures the target substance 21 contained in the liquid film 7. As a result, the chemical sensor device 10 can accurately detect the target substance 21.

The detection site 6b is a nucleic acid. The detection site 6b may be a deoxyribonucleic acid (DNA) or a ribonucleic acid (RNA), or may be an artificial nucleic acid such as LISA. The nucleic acid may be a single-stranded nucleic acid or a double-stranded nucleic acid, and is preferably a single-stranded nucleic acid. The detection site 6b functions as an aptamer, and binds to the target substance 21 in a state of taking a predetermined conformation. A group constituting the immobilized site 6a (for example, a polycyclic aromatic group described below) may be modified with a phosphoric acid group, sugar and/or a base at any end of the nucleic acid, and the group constituting the immobilized site 6a may be modified in a portion other than the end of the nucleic acid as long as the property of binding to the target substance 21 is secured.

The nucleic acid compound 6 is preferably one in which the nucleic acid is modified with a polycyclic aromatic group. The nucleic acid compound 6 can be firmly immobilized on the sensitive film 2 by an interaction (n-n interaction) between the n bond of the polycyclic aromatic group modified in the nucleic acid and the n bond of a substance (for example, graphene) constituting the sensitive film 2. That is, the polycyclic aromatic group functions as the immobilized site 6a of the nucleic acid compound 6. However, the immobilized site 6a does not necessarily need to be adsorbed to the sensitive film 2 by the n-n interaction. The immobilized site 6a can be covalently bonded or electrostatically bonded to the sensitive film 2, or can be adsorbed to the sensitive film 2 by any other methods depending on the design.

Examples of the polycyclic aromatic group include a biphenylyl group, a terphenylyl group, a naphthyl group, an anthryl group, a phenanthryl group, a fluorenyl group, an indenyl group, a pyrenyl group, a perylenyl group, a fluoranthenyl group, a triphenylenyl group, a quinolyl group, an isoquinolyl group, a benzofuranyl group, a benzothienyl group, an indolyl group, a carbazolyl group, a benzoxazolyl group, a benzothiazolyl group, a quinoxalyl group, a benzimidazolyl group, a dibenzofuranyl group, a dibenzothienyl group, a xanthene ring, and an indole ring. Among them, a pyrenyl group is preferred. In the nucleic acid compound 6 shown in FIG. 2, the nucleic add is modified with a pyrenyl group, and a portion of the pyrenyl group functions as the immobilized site 6a of the nucleic add compound 6.

The polycyclic aromatic group may further have a substituent.

A labeled molecule 6c can be modified at an end opposite to the end at which the immobilized site 6a of the nucleic acid (detection site 6b) is modified, as necessary. As the labeled molecule 6c, a molecule having a strong charge or a fluorescent dye can be used. When the nucleic acid is modified with the labeled molecule 6c, the structural change of the nucleic acid associated with the capture of the target substance 21 can be detected not only by a negative charge of the main skeleton of the nucleic acid but also by a signal obtained from the labeled molecule 6c.

When the nucleic acid captures the target substance 21 at the time of detection of the target substance 21, the nucleic acid compound 6 and the target substance 21 form a complex, and the conformation of the nucleic acid compound 6 changes. When the target substance 21 is removed from the complex, the conformation of the nucleic acid compound 6 returns to the original structure. That is, a change in the conformation of the nucleic acid compound 6 is reversible. Therefore, even when the target substance 21 in the specimen liquid 111 is detected and then the target substance 21 is removed to form the liquid film 7 including a new specimen liquid 111, the target substance 21 can be detected again in the same manner. That is, the detection of the target substance 21 described above can be repeatedly performed.

On the surface 2a of the sensitive film 2, in addition to the nucleic acid compound 6, the blocking agent 8 is disposed so as to cover the surface 2a, The blocking agent 8 in the embodiment is a phosphoric acid derivative.

The phosphoric acid derivative is preferably one or more selected from the group consisting of a phosphoric acid alkyl ester and a phosphoric acid alkyl ester oligomer.

The phosphoric acid alkyl ester is preferably one represented by general formula: OP(OR¹)(OR²)(OR³). In the formula, each of R¹, R², and R³ is preferably independently selected from a C1 to C10 aliphatic group (having no aromatic ring), a C6 to C20 aryl group, a C7 to C30 alkylaryl group, and a C7 to C30 arylalkyl. Specific examples of the phosphoric acid alkyl ester represented by general formula: OP(OR¹)(OR²)(OR³) include tributylphosphate and 2-ethylhexyldiphenylphosphate.

The phosphoric acid alkyl ester oligomer is a polymer of a phosphoric acid alkyl ester. Specific examples of the phosphoric acid alkyl ester oligomer include a poly-d-spacer (DNA having no base).

The phosphoric acid alkyl ester and the phosphoric acid alkyl ester oligomer are substances in which the whole or a part of three hydrogen atoms of phosphoric acid (H₃PO₄) is replaced with an aliphatic group or the like, and thus are less likely to be ionized and charged as compared with phosphoric acid. Therefore, it is difficult to interfere a signal of the negative charge of the phosphoric acid ester moiety which is the main skeleton of the nucleic acid compound 6. As a result, a change in the conformation of the nucleic acid compound 6 is easily detected, and the target substance 21 is easily detected.

Since the phosphoric acid alkyl ester and the phosphoric acid alkyl ester oligomer are less likely to be ionized, the phosphoric acid alkyl ester and the phosphoric acid alkyl ester oligomer have higher hydrophobicity than phosphoric acid, and when the solvent of the specimen liquid 111 is water or the like, solubility in the specimen liquid 111 decreases. Therefore, the phosphoric add alkyl ester and the phosphoric add alkyl ester oligomer are less likely to be eluted into the specimen liquid 111 when the target substance 21 is detected, and the adsorption power on the surface 2a of the sensitive film 2 is increased.

The liquid film 7 is disposed on the surface 2a of the sensitive film 2 so as to cover the nucleic acid compound 6 and the blocking agent 8. The liquid film 7 includes the specimen liquid 111 capable of containing the target substance 21, and serves as a medium for carrying the target substance 21 to the nucleic acid compound 6. Since the liquid film 7 is disposed so as to cover the nucleic acid compound 6, denaturation or damage of the nucleic acid compound 6 due to drying can be prevented.

The liquid film 7 has a thickness of, for example, 0.5 μm or more and 10.0 μm or less. The thickness of the liquid film 7 refers to, for example, the shortest distance between the surface 2a of the sensitive film 2 and the surface of the liquid film 7 in FIG. 1. When the thickness of the liquid film 7 is less than 0.5 μm, the target substance 21 that can be contained in the specimen liquid 111 easily reaches the nucleic acid compound 6, and the sensitivity of the chemical sensor can be improved; however, there is a possibility that the liquid film 7 is dried and denaturation or damage of the nucleic acid compound 6 cannot be prevented. On the other hand, when the thickness of the liquid film 7 exceeds 10.0 μm, the target substance 21 that can be contained in the specimen liquid 111 is less likely to reach the nucleic acid compound 6, and thus the sensitivity of the chemical sensor may be deteriorated. The thickness of the liquid film 7 is preferably, for example, 0.5 μm or more and 5.0 μm or less.

Alternatively, a ceiling may be formed above the liquid film 7, and the liquid film 7 may be a flow path integrated with the liquid supply mechanism 110 and the liquid discharge mechanism 120. In this case, there is no restriction on the thickness of the liquid film 7.

The target substance 21 is a substance that can be contained in the specimen liquid 111, and is a substance that can bind to the nucleic acid compound 6. Most of the target substances 21 are substances that can be dissolved in the specimen liquid 111.

The target substance 21 is, for example, a substance serving as an index of a property, a state, and/or a change in property and state of an organism, a material, an environment, or the like from which a sample is derived, and the target substance 21 is not particularly limited. For example, when the sample is a biological sample, the property and state are health conditions of an organism from which the sample is derived. The health conditions are, for example, presence or absence of a disease in an organism, a characteristic, a severity course and/or an effect or side effect on a medicine, and the like. Alternatively, the target substance 21 is a type or the like of virus or microorganism in which an organism is infected or which is contained in a sample.

The target substance 21 is, for example, a nucleic acid, a protein, an endocrine substance, a cell, a blood cell, a virus, a microorganism, an organic compound, an inorganic compound, or a low molecular weight compound such as an odor component or a volatile organic chemical substance.

The specimen liquid 111 constitutes the liquid film 7 and is a liquid sample to be analyzed capable of containing the target substance 21, As the specimen liquid 111, a liquid diluted with a solvent may be used. The solvent may be an inorganic solvent or an organic solvent. Examples of the solvent that can be used include water, physiological water, an ionic liquid, a PB buffer, a PBS buffer, an HEPES buffer, a Tris buffer, DMF, DMSO, alcohol, and a mixture of any of these. The liquid film 7 may further contain a solute (a non-target substance 22) such as a pH adjuster, a preservative, and a stabilizer.

As described above, since the chemical sensor device 10 uses the sensitive film 2, the nucleic acid compound 6, and the specific blocking agent 8 as the detection mechanism of the target substance 21, the chemical sensor device 10 can detect the target substance 21 that can be contained in the specimen liquid 111 with high sensitivity.

When the chemical sensor device 10 includes the liquid supply mechanism 110 and the liquid discharge mechanism 120, after the target substance 21 in the specimen liquid 111 is detected, the liquid film 7 is removed by the liquid discharge mechanism 120, the specimen liquid 111 is newly supplied from the liquid supply mechanism 110, whereby the liquid film 7 can be formed again. Alternatively, a flow of the liquid from one end to the other end of the liquid film 7 can be generated to form a new liquid film 7. As a result, the chemical sensor device 10 can repeatedly detect the target substance 21.

The chemical sensor device 10 described above has a configuration of a graphene field effect transistor (hereinafter, also referred to as “graphene FET”), but is not limited to the graphene FET. In the case of using the nucleic acid compound 6, for example, the chemical sensor device 10 may have a configuration of another charge detection element such as ion sensitive FET (ISFET), a surface plasmon resonance (SPR) element, a surface acoustic wave (SAW) element, a film bulk acoustic resonance (FGAR) element, a quartz crystal microbalance (QCM) element, a micro electro mechanical system (MEMS) cantilever element, or the like.

Hereinafter, a method for detecting a target substance using the chemical sensor device of the embodiment will be described.

A method for detecting a target substance includes, in the stated order, a first step of exposing a sensitive film to a first solution containing a phosphoric acid derivative, a second step of exposing the sensitive film to a second solution containing a nucleic acid compound, and a third step of exposing the sensitive film to an specimen liquid capable of containing a target substance.

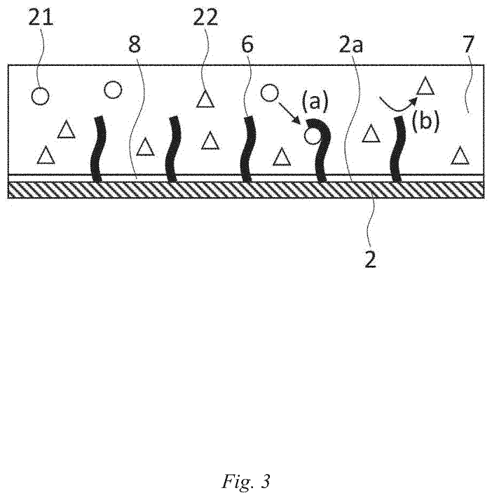

Hereinafter, the principle of detecting a target substance by performing each of the above steps will be described. The third step will be described with reference to FIG. 3.

In the first step, the sensitive film 2 is exposed to the first solution containing a phosphoric acid derivative. For example, the first solution is supplied by dropping with a pipette, an inkjet, a dispenser, or the like, a liquid film including the first solution is provided on the surface 2a of the sensitive film 2, and the first solution and the sensitive film 2 are brought into contact with each other. The phosphoric acid derivative functions as a blocking agent that occupies an adsorption site on the surface 2a of the sensitive film 2.

When the first solution and the sensitive film 2 come into contact with each other, the phosphoric acid derivative contained in the first solution is adsorbed to the adsorption site on the surface 2a of the sensitive film 2. The whole or most of the adsorption site suitable for adsorption of the phosphoric acid derivative can be occupied by the phosphoric acid derivative, and the surface 2a of the sensitive film 2 can be covered with the phosphoric acid derivative. As a result, even when the sensitive film 2 is exposed to the second solution containing the nucleic acid compound 6 in the second step described below, the phosphoric acid derivative is already adsorbed to the adsorption site on the surface 2a of the sensitive film 2, so that it is possible to prevent the nucleic acid compound 6 from being adsorbed (non-specifically adsorbed) to the adsorption site on the surface 2a of the sensitive film 2 at the detection site 6b.

The concentration of the phosphoric acid derivative in the first solution can be appropriately adjusted depending on the type of the phosphoric acid derivative, and is, for example, preferably 0.01 μmol/L or more and 10 mmol/L or less, more preferably 0.1 μmol/L or more and 1 mmol/L or less, and further preferably 1 μmol/L or more and 100 μmol/L or less.

The solvent in the first solution may be an inorganic solvent or an organic solvent. Examples of the solvent that can be used include water, physiological water, an ionic liquid, a PB buffer, a PBS buffer, an HEPES buffer, a Tris buffer, DMF, DMSO, alcohol, and a mixture of any of these. The first solution may further contain a solute such as a pH adjuster, a preservative, and a stabilizer.

The method for detecting a target substance according to the embodiment may include a plurality of first steps. When there are a plurality of first steps, the first solutions may be the same as or different from each other. For example, the type and/or concentration of the phosphoric acid derivative may be different in the first solution used in each first step.

In the second step, the sensitive film 2 is exposed to the second solution containing the nucleic acid compound 6. For example, as in the first step, the second solution is supplied by dropping with a pipette, an inkjet, a dispenser, or the like, and a liquid film including the second solution is provided on the surface 2a of the sensitive film 2 covered with the phosphoric acid derivative. The second solution is in contact with the sensitive film 2 via the phosphoric add derivative.

On the surface 2a of the sensitive film 2, the adsorption site to which the phosphoric acid derivative binds and the adsorption site to which the immobilized site 6a of the nucleic acid compound 6 binds may be different from each other. Therefore, even when the surface 2a of the sensitive film 2 is covered with the phosphoric acid derivative, the nucleic acid compound 6 contained in the second solution can be adsorbed onto the surface 2a of the sensitive film 2.

In the second step, the immobilized site 6a of the nucleic acid compound 6 contained in the second solution binds to the adsorption site on the surface 2a of the sensitive film 2, so that the nucleic acid compound 6 is immobilized on the surface 2a of the sensitive film 2. Then, the detection site 6b of the nucleic acid compound 6 is not adsorbed onto the surface 2a of the sensitive film 2 and is located on the liquid film 7 side. Therefore, in the third step described below, the detection site 6b of the nucleic acid compound 6 can capture the target substance 21 without any trouble, and the target substance 21 can be detected.

When the second step is executed without passing through the first step, since the surface 2a of the sensitive film 2 is not covered with the blocking agent 8, not only the immobilized site 6a of the nucleic acid compound 6 is adsorbed onto the surface 2a of the sensitive film 2, but also the detection site 6b of the nucleic acid compound 6 is adsorbed onto the surface 2a of the sensitive film 2 (non-specific adsorption). Then, the binding (specific adsorption) between the detection site 6b of the nucleic acid compound 6 and the target substance 21 is less likely to occur, or even if the binding occurs, the conformation of the nucleic acid compound 6 is less likely to change in some cases. As a result, the target substance 21 may not be accurately detected. In the embodiment, the surface 2a of the sensitive film 2 is covered with the phosphoric acid derivative in advance before the nucleic acid compound 6 is immobilized on the surface 2a of the sensitive film 2, so that the binding (non-specific adsorption) between the detection site 6b of the nucleic acid compound 6 and the sensitive film 2 can be prevented and the nucleic acid compound 6 can be bonded onto the surface 2a of the sensitive film 2 only with the immobilized site 6a.

The concentration of the nucleic add compound 6 in the second solution can be appropriately adjusted depending on the type of the nucleic acid compound 6, and is, for example, preferably 1 nmol/L or more and 100 μmol/L or less and more preferably 10 nmol/L. or more and 10 μmol/L. or less.

The solvent in the second solution may be an inorganic solvent or an organic solvent, Examples of the solvent that can be used include water, physiological water, an ionic liquid, a PB buffer, a PBS buffer, an HEPES buffer, a Tris buffer, DMF, DMSO, alcohol, and a mixture of any of these. The second solution may further contain a solute such as a pH adjuster, a preservative, and a stabilizer.

The method for detecting a target substance according to the embodiment may include a plurality of second steps. When there are a plurality of second steps, the second solutions may be the same as or different from each other. For example, the type and/or concentration of the nucleic add compound 6 may be different in the second solution used in each second step.

In the third step, the sensitive film 2 in which the phosphoric add derivative and the nucleic acid compound 6 are immobilized on the surface 2a is exposed to the specimen liquid 111. The state of the chemical sensor device 10 at this time is shown in FIG. 3. When the target substance 21 is contained in the specimen liquid 111, the target substance 21 moves in the liquid film 7 including the specimen liquid 111 and binds to the nucleic add compound 6 ((a) in FIG. 3). On the other hand, the non-target substance 22 (contaminant) does not bind to the nucleic add compound 6 ((b) in FIG. 3). When the target substance 21 and the nucleic add compound 6 are bonded to each other, the conformation of the nucleic add compound 6 is changed ((a) in FIG. 3), and the physical properties of the sensitive film 2 to which the nucleic add compound 6 is adsorbed are changed. The physical properties are, for example, electric resistance of the sensitive film 2, and the like.

After the third step, a change in physical properties is detected as a change in electrical signal. The electrical signal is, for example, a current value, a potential value, an electric resistance value, an impedance value, or the like. The change in electrical signal is, for example, an increase, a decrease, or a disappearance in electrical signal, a change in integrated value within a specific time, or the like. In the case of using the graphene FET described above, the change in physical properties can be detected as, for example, a change in source-drain current value when a constant voltage is applied as a gate voltage and a drain voltage. Alternatively, the change in physical properties may be detected as a change in gate voltage value when the source-drain current value reaches a predetermined value. The information on the change in electrical signal is transmitted to, for example, an electrically connected data processing unit or the like, stored, and processed.

The presence or absence or the amount of the target substance 21 in the specimen liquid 111 can be determined from the detection result. For example, it may be determined that the target substance 21 is present in the specimen liquid 111 when a change in electrical signal occurs, and it may be determined that the target substance 21 is not present when no change occurs. It may be determined that the target substance 21 is present when the electrical signal changes more than a preset threshold value, and it may be determined that the target substance 21 is not present when the change is less than the threshold value. Such a threshold value can be obtained, for example, by subjecting a liquid sample known to contain the target substance 21 to analysis of a chemical sensor to obtain a change value of an electrical signal. Alternatively, the amount of the target substance 21 may be determined by the amount of change. In this case, a calibration curve of the amount of change with respect to the concentration of the target substance 21 is created using a liquid sample in which the concentration of the target substance 21 is known, and the amount of the target substance 21 may be determined by comparing with the calibration curve.

According to the steps described above, the method for detecting the target substance 21 of the embodiment can detect the target substance 21 that can be contained in the specimen liquid 111 with high sensitivity.

The method for detecting a target substance according to the embodiment may include other steps in addition to the first step, the second step, and the third step. For example, the method for detecting a target substance may include a step of exposing the sensitive film to a liquid other than the first solution, the second solution, and the specimen liquid between the first step and the second step and/or between the second step and the third step.

When the chemical sensor device 10 of the embodiment includes the liquid supply mechanism 110 and the liquid discharge mechanism 120, after the target substance 21 in the specimen liquid 111 is detected, the liquid film 7 is removed by the liquid discharge mechanism 120, the specimen liquid 111 is newly supplied from the liquid supply mechanism 110, whereby the liquid film 7 can be formed again. Alternatively, a flow of the liquid from one end to the other end of the liquid film 7 can be generated to form a new liquid film 7. Therefore, it becomes easy to repeatedly perform the detection of the target substance 21 described above.

The method for detecting a target substance may be performed by a device that automatically performs each step. Such a device includes, for example, the chemical sensor device 10, a detection unit that converts a change in physical properties of the sensitive film 2 into a change in electrical signal, a data processing unit that stores and processes information on the electrical signal obtained from the detection unit, and a control unit that controls operations of the detection unit and the data processing unit. The operation of each step may be executed by an input of an operator of the device, or may be executed by a program included in the control unit.

According to the method for detecting the target substance 21 using the chemical sensor device 10 of the embodiment, since the nucleic acid compound 6 that specifically binds to the target substance 21 is used, it is possible to prevent the non-target substance 22 from being detected. Therefore, even under conditions that the composition of a substance contained in the specimen liquid 111 is different, the target substance 21 can be detected without being affected by the non-target substance 22.

### EXAMPLES

### Example 1

A device in which the liquid supply mechanism 110 and the liquid discharge mechanism 120 were removed from the chemical sensor device 10 shown in FIG. 1 was manufactured by the following procedure.

First, a composite device, which includes a substrate, a sensitive film (graphene film) provided on the substrate, a source electrode (gold electrode, titanium or chromium as an adhesion layer on a base) connected to one end of the sensitive film, a drain electrode (gold electrode, titanium or chromium as an adhesion layer on a base) connected to the other end of the sensitive film, a protective insulating film formed on the source electrode and the drain electrode and opened so that a part of the sensitive film is exposed, and a wall portion erected on the protective insulating film, was prepared.

A gate electrode (silver/silver chloride electrode) was disposed on the upper part of the composite device, the gate voltage was set to 0 my, and 5 mV was applied between the source electrode and the drain electrode. While the drain current was measured, a solution in which 1 mass % of ethanol was contained in a buffer solution (1 mmol/L of HEPES and 150 mmol/L of potassium chloride) (hereinafter, referred to as “ethanol-containing buffer solution) was added dropwise to the sensitive film by pipetting.

After the drain current was stabilized, the ethanol-containing buffer solution on the surface of the sensitive film was replaced with an ethanol-containing buffer solution having the same composition by pipetting, and it was confirmed that noise due to the pipetting was small.

Thereafter, the ethanol-containing buffer solution on the surface of the sensitive film was replaced with a 1 μmol/L tributylphosphate solution (prepared by dissolving tributylphosphate in ethanol to obtain a 100 μmol/L solution, and then diluting the solution 100 times with the above-described buffer solution) by pipetting. At this time, it was confirmed that the drain current was greatly reduced. That is, it was suggested that tributylphosphate was adsorbed to the surface of the sensitive film.

After the change in the drain current was stabilized, the 1 μmol/L tributylphosphate solution on the surface of the sensitive film was replaced with a 10 μmol/L tributylphosphate solution (prepared by dissolving tributylphosphate in ethanol to obtain a 1000 μmol/L solution, and then diluting the solution 100 times with the above-described buffer solution). At this time, it was confirmed that the drain current was further reduced. That is, it was suggested that tributylphosphate was additionally adsorbed to the surface of the sensitive film.

Thereafter, the 10 μmol/L tributylphosphate solution on the surface of the sensitive film was replaced with the above-described ethanol-containing buffer solution by pipetting. At this time, the drain current was increased. The increase amount of the drain current was about half as compared with the decrease amount of the drain current due to the replacement with the 10 μmol/L tributylphosphate solution, and was about quarter as compared with the total decrease amount of the drain current due to the replacement twice with the 1 μmol/L tributylphosphate solution and the 10 μmol/L tributylphosphate solution. That is, it was suggested that most of tributylphosphate remained adsorbed to the surface of the sensitive film even when replacement was performed with the ethanol-containing buffer solution.

Thereafter, the ethanol-containing buffer solution on the surface of the sensitive film was replaced twice with physiological saline (150 mmol/L sodium chloride aqueous solution) by pipetting to remove free tributylphosphate.

After the change in the drain current was settled, the physiological saline on the surface of the sensitive film was replaced with a 100 nmol/L DNA solution (a solution obtained by dissolving tricontamer of adenine in physiological saline) by pipetting. At this time, the drain current did not change. Since the DNA used in Example 1 was not modified with a group corresponding to the immobilized site, the drain current did not change, suggesting that the DNA main body corresponding to the detection site was not adsorbed to the sensitive film. That is, it was suggested that non-specific adsorption between the DNA and the sensitive film was suppressed.

Thereafter, the DNA solution on the surface of the sensitive film was replaced with physiological saline by pipetting. At this time, the drain current did not change.

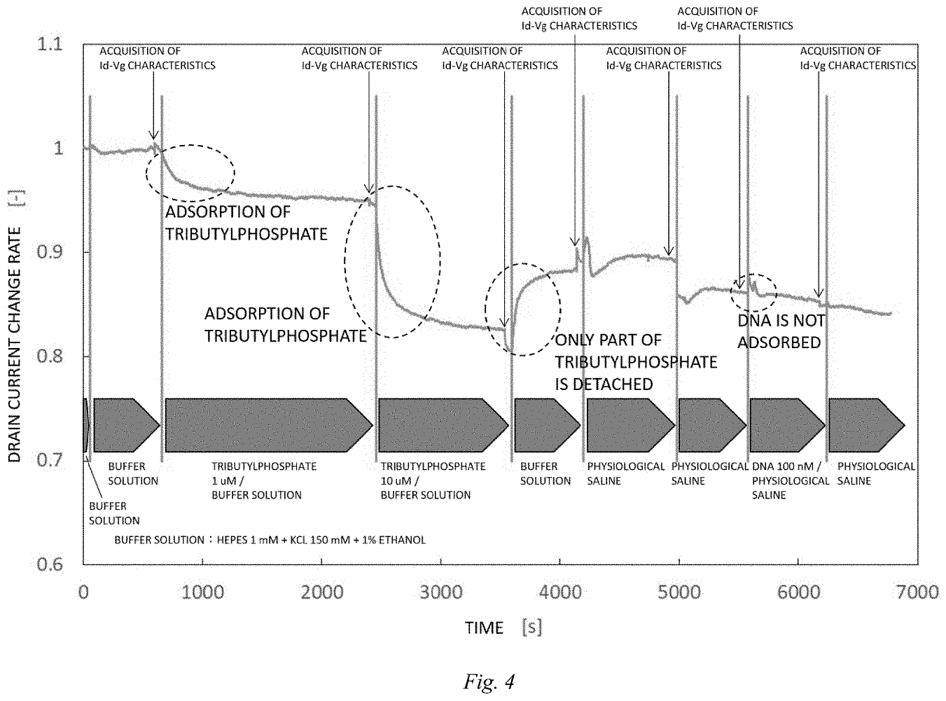

In this way, a chemical sensor device (graphene FET) of Example 1 was manufactured. A measurement result of the drain current change rate at the time of manufacturing the chemical sensor device of Example 1 is shown in FIG. 4.

The drain current change rate is a value obtained by dividing the measured value of the drain current at each time by the average value of the drain current in the initial 50 seconds.

In the middle of manufacturing the chemical sensor device of Example 1, the measurement of the time-dependent change of the drain current was temporarily interrupted as necessary, and the gate voltage was swept to acquire Id-Vg characteristics. When the measurement of the drain current is restarted after the acquisition of the Id-Vg characteristics, a trace of changing the gate voltage appears as a slight nose at the time of restart. FIG. 4 shows a plurality of measurement results of time-dependent change interrupted in the middle for acquisition of the Id-Vg characteristics in order.

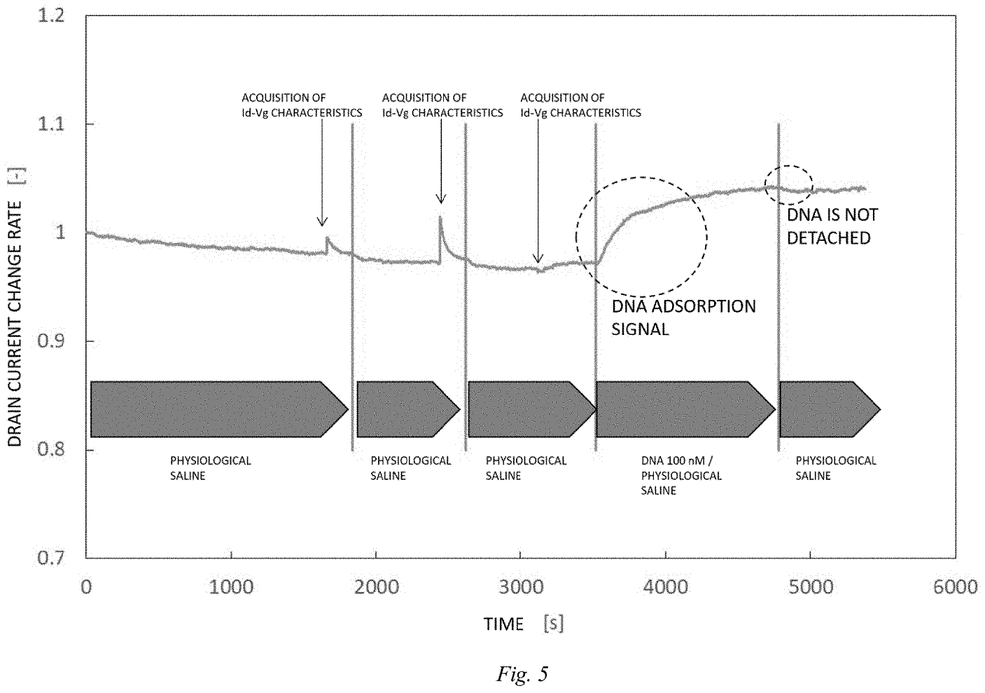

The same applies to Comparative Examples 1 to 5 and FIGS. 5 to 9 described below.

### Comparative Example 1

The same composite device as that of Example 1 was prepared.

While the drain current of the composite device was measured, physiological saline was added dropwise to the sensitive film by pipetting. The gate voltage was set to 0 mV, and 5 mV was applied between the source electrode and the drain electrode.

After the drain current was stabilized, the physiological saline on the surface of the sensitive film was replaced with physiological saline having the same composition by pipetting, and it was confirmed that noise due to the pipetting did not occur.

Thereafter, the physiological saline on the surface of the sensitive film was replaced again with physiological saline having the same composition by pipetting.

Thereafter, the physiological saline on the surface of the sensitive film was replaced with a 100 nmol/L single-stranded DNA solution (a solution obtained by dissolving a DNA having a base sequence of 5′-GACAA GGAAA ATCCT TCAAT GAAGT GGGTC-3′ in physiological saline) by pipetting. At this time, the drain current was increased. That is, it was suggested that non-specific adsorption between the single-stranded DNA and the sensitive film occurred.

Thereafter, the single-stranded DNA solution on the surface of the sensitive film was replaced with physiological saline by pipetting. At this time, the drain current did not change, That is, it was suggested that the single-stranded DNA adsorbed to the sensitive film was not easily detached.

In this way, a chemical sensor device (graphene FET) of Comparative Example 1 was manufactured. A measurement result of the drain current change rate at the time of manufacturing the chemical sensor device of Comparative Example 1 is shown in FIG. 5,

### Comparative Example 2

The same composite device as that of Example 1 was prepared.

While the drain current of the composite device was measured, a buffer solution (an aqueous solution of 1 mmol/L, of HEPES and 1 mmol/L. of potassium chloride) was added dropwise to the sensitive film by pipetting. The gate voltage was set to 75 my, and 5 mV was applied between the source electrode and the drain electrode.

After the drain current was stabilized, the buffer solution on the surface of the sensitive film was replaced with a 1 μmol/L peptide nucleic acid solution (a solution obtained by dissolving a modified product obtained by modifying the nucleoside base sequence of TCTTCCTTTTTT with a lysine pentamer at the N-terminal side and a polyethylene glycol at the C-terminal side in the above-described buffer solution) by pipetting. At this time, it was confirmed that the drain current was greatly reduced. That is, it was suggested that peptide nucleic acid (PNA) was adsorbed to the surface of the sensitive film.

Thereafter, the peptide nucleic acid solution on the surface of the sensitive film was replaced with the above-described buffer solution by pipetting. At this time, the drain current did not change. That is, it was suggested that the PNA adsorbed to the sensitive film was not easily detached.

The gate voltage was changed to 0 my. At this time, it was confirmed that the drain current was increased. This is because the Fermi level of the sensitive film changed and hole injection occurred.

Thereafter, the buffer solution on the surface of the sensitive film was replaced with physiological saline by pipetting. At this time, it was confirmed that the drain current was reduced. This is because the potential difference between the gate electrode (silver/silver chloride electrode) and the physiological saline was changed by an increase in concentration of chloride ion.

Thereafter, the physiological saline on the surface of the sensitive film was replaced again with physiological saline having the same composition by pipetting.

After it was confirmed that the change in the drain current was small, the physiological saline on the surface of the sensitive film was replaced with a 100 nmol/L single-stranded DNA solution (a solution obtained by dissolving tricontamer of thymine in physiological saline) by pipetting. At this time, the drain current was increased, That is, it was suggested that non-specific adsorption between the single-stranded DNA and the sensitive film occurred.

Thereafter, the single-stranded DNA solution on the surface of the sensitive film was replaced with physiological saline by pipetting. At this time, the drain current did not change, That is, it was suggested that the single-stranded DNA adsorbed to the sensitive film was not easily detached.

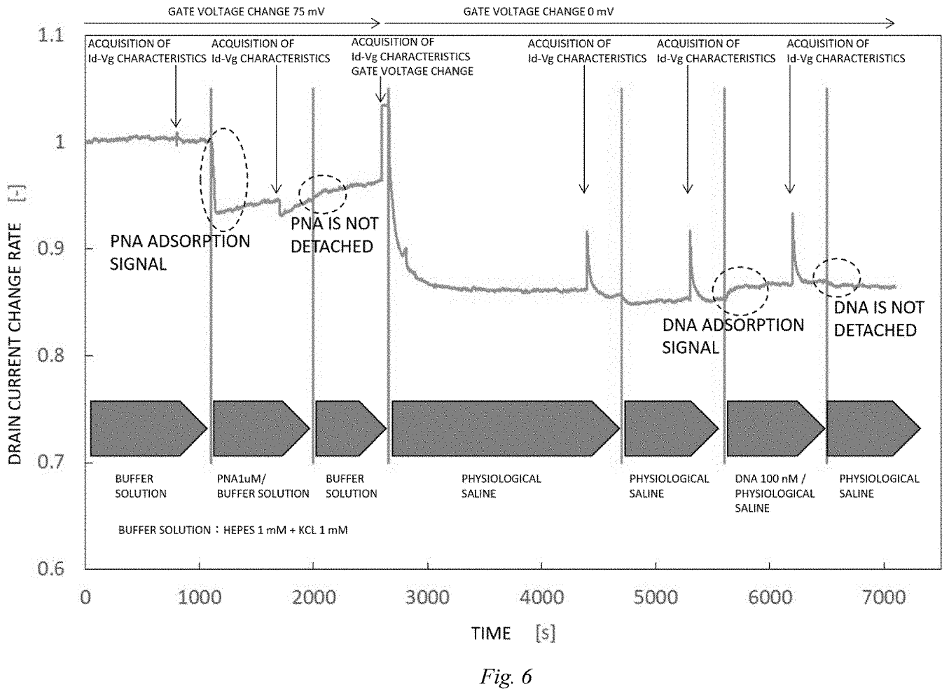

In this way, a chemical sensor device (graphene FET) of Comparative Example 2 was manufactured. A measurement result of the drain current change rate at the time of manufacturing the chemical sensor device of Comparative Example 2 is shown in FIG. 6.

### Comparative Example 3

The same composite device as that of Example 1 was prepared.

While the drain current of the composite device was measured, physiological saline was added dropwise to the sensitive film by pipetting. The gate voltage was set to 0 mV, and 5 mV was applied between the source electrode and the drain electrode.

After the drain current was stabilized, the physiological saline on the surface of the sensitive film was replaced with physiological saline having the same composition by pipetting, and it was confirmed that noise due to the pipetting was small to such an extent that detection of an adsorption signal of a double-stranded DNA was not inhibited.

Thereafter, the physiological saline on the surface of the sensitive film was replaced again with physiological saline having the same composition by pipetting.

Thereafter, the physiological saline on the surface of the sensitive film was replaced with a double-stranded DNA solution (a solution obtained by dissolving tricontamer of thymine and tricontamer of adenine that is a complementary chain thereof in physiological saline) adjusted to have a single-stranded concentration of 100 nmol/L by pipetting. At this time, the drain current was increased, That is, it was suggested that non-specific adsorption between the double-stranded DNA and the sensitive film occurred.

Thereafter, the double-stranded DNA solution on the surface of the sensitive film was replaced with physiological saline by pipetting. At this time, the drain current did not change. That is, it was suggested that the double-stranded DNA adsorbed to the sensitive film was less likely to be detached.

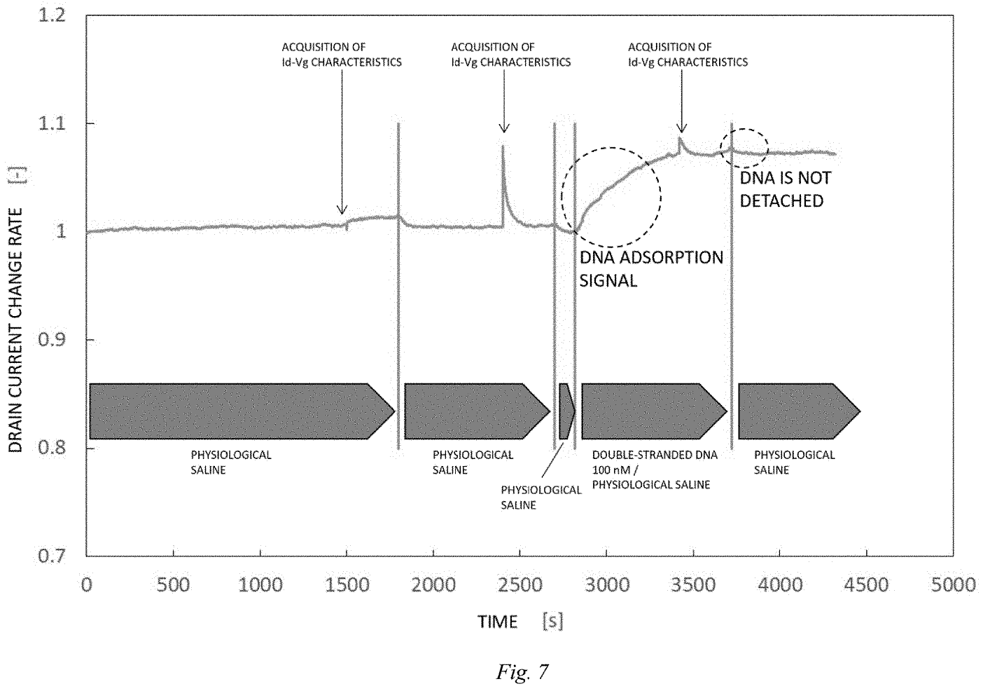

In this way, a chemical sensor device (graphene FET) of Comparative Example 3 was manufactured. A measurement result of the drain current change rate at the time of manufacturing the chemical sensor device of Comparative Example 3 is shown in FIG. 7.

### Comparative Example 4

The same composite device as that of Example 1 was prepared.

While the drain current of the composite device was measured, physiological saline was added dropwise to the sensitive film by pipetting. The gate voltage was set to 0 mV, and 5 mV was applied between the source electrode and the drain electrode.

After the drain current was stabilized, the physiological saline on the surface of the sensitive film was replaced with physiological saline having the same composition by pipetting. This replacement operation was repeated twice, and it was confirmed that noise due to the pipetting was small to such an extent that detection of an adsorption signal of the double-stranded DNA was not inhibited.

Thereafter, the physiological saline on the surface of the sensitive film was replaced with a double-stranded DNA solution (a solution obtained by dissolving a self-dimer of DNA having a base sequence of 5′-GGAAA GAGAA TATTC TCTTT CC-3′ in physiological saline) adjusted to have a single-stranded concentration of 100 nmol/L by pipetting. At this time, the drain current was increased. That is, it was suggested that non-specific adsorption between the double-stranded DNA and the sensitive film occurred.

Thereafter, the double-stranded DNA solution on the surface of the sensitive film was replaced with physiological saline by pipetting. At this time, the drain current did not change, That is, it was suggested that the double-stranded DNA adsorbed to the sensitive film was less likely to be detached.

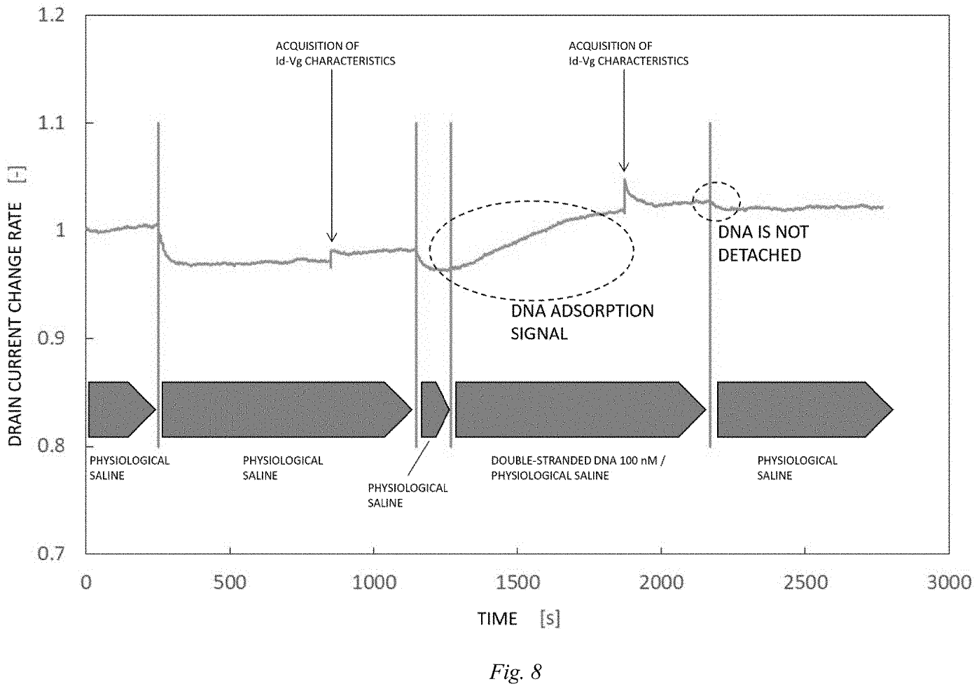

In this way, a chemical sensor device (graphene FET) of Comparative Example 4 was manufactured. A measurement result of the drain current change rate at the time of manufacturing the chemical sensor device of Comparative Example 4 is shown in FIG. 8.

### Comparative Example 5

The same composite device as that of Example 1 was prepared.

While the drain current of the composite device was measured, a buffer solution (an aqueous solution of 1 mmol/L of HEPES and 150 mmol/L of potassium chloride) was added dropwise to the sensitive film by pipetting. The gate voltage was set to 0 my, and 5 mV was applied between the source electrode and the drain electrode.

After the drain current was stabilized, the buffer solution on the surface of the sensitive film was replaced with a D-PBS(−) solution (an aqueous solution of 2.7 mmol/L of potassium chloride, 136.9 mmol/L of sodium chloride, 1.5 mmol/L of potassium dihydrogen phosphate, and 8.1 mmol/L of disodium hydrogen phosphate) by pipetting. At this time, it was confirmed that the drain current was increased. This is because the concentration of chloride ions slightly decreased.

Thereafter, the mixed solution on the surface of the sensitive film was replaced with the above-described buffer solution by pipetting. At this time, the drain current was reduced and returned to the original value. This is because the concentration of chloride ions returned to the original concentration.

Thereafter, the buffer solution on the surface of the sensitive film was replaced with physiological saline by pipetting. At this time, it was confirmed that the drain current was reduced. This is considered to be because the solution was replaced with a solution having no pH buffering ability.

Thereafter; the physiological saline on the surface of the sensitive film was replaced again with physiological saline having the same composition by pipetting.

After it was confirmed that the change in the drain current was small, the physiological saline on the surface of the sensitive film was replaced with a 100 nmol/L single-stranded DNA solution (a solution obtained by dissolving tricontamer of thymine in physiological saline) by pipetting. At this time, the drain current was slightly reduced for a moment, but immediately increased and returned to the original value. The change in the drain current at this time can be regarded as noise at the time of pipetting, but even if it was a signal associated with DNA adsorption, the change was very small. In Comparative Example 5, the change in the drain current due to the replacement with the single-stranded DNA solution was suppressed as compared with Comparative Examples 1 to 4. That is, it was suggested that non-specific adsorption between the single-stranded DNA and the sensitive film was suppressed.

Thereafter, the single-stranded DNA solution on the surface of the sensitive film was replaced with physiological saline by pipetting. At this time, a slight decrease in the drain current suggested a possibility that a very small amount of single-stranded DNA was weakly adsorbed. These results suggested that the water-soluble phosphate ion contained in the D-PBS(−) solution has a certain effect of inhibiting non-specific adsorption of the single-stranded DNA to the sensitive film. However, it was also suggested that such an effect was slightly inferior in Comparative Example 5 as compared with Example 1.

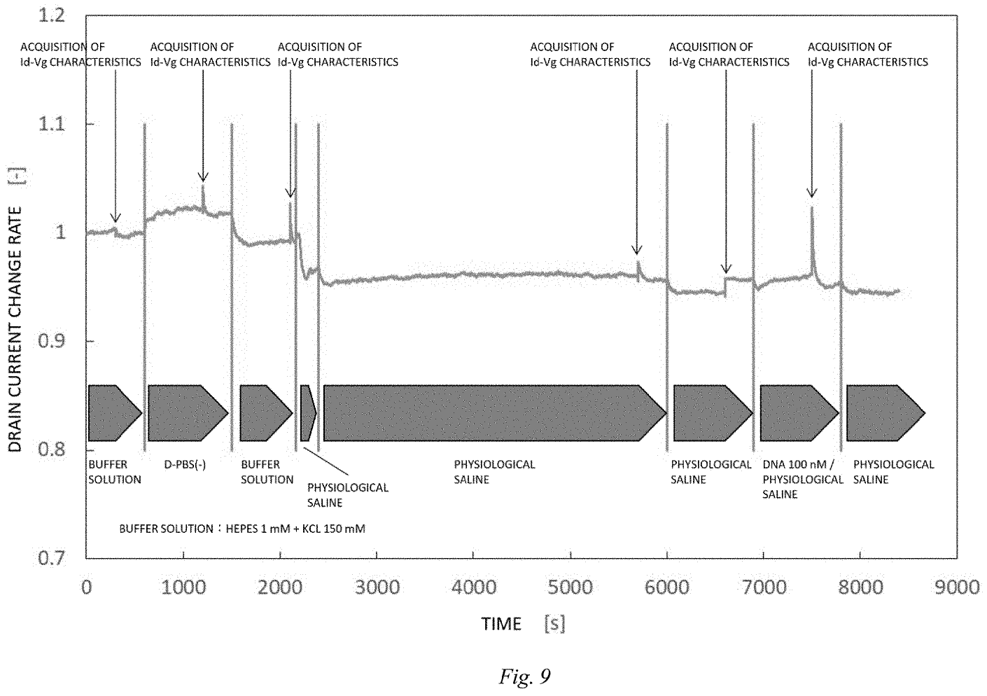

In this way, a chemical sensor device (graphene FET) of Comparative Example 5 was manufactured. A measurement result of the drain current change rate at the time of manufacturing the chemical sensor device of Comparative Example 5 is shown in FIG. 9,

From the results of Example 1 and Comparative Examples 1 to 5, it was found that non-specific adsorption of the DNA to the sensitive film can be suppressed by the chemical sensor device according to the embodiment of the present invention.

While certain embodiments have been described, these embodiments have been presented by way of example only, and are not intended to limit the scope of the inventions, Indeed, the novel methods and systems described herein may be embodied in a variety of other forms; furthermore, various omissions, substitutions and changes in the form of the methods and systems described herein may be made without departing from the sprit of the inventions. The accompanying claims and their equivalents are intended to cover such forms or modifications as would fail within the scope and sprit of the invention.

1. A chemical sensor device comprising:
a sensitive film;
a nucleic add compound that is immobilized on said sensitive film; and
a phosphoric acid derivative that is immobilized on a surface of said sensitive film on which said nucleic acid compound is immobilized.

2. The chemical sensor device according to claim 1, wherein said phosphoric acid derivative is one or more selected from the group consisting of a phosphoric acid alkyl ester and a phosphoric acid alkyl ester oligomer.

3. The chemical sensor device according to claim 2, wherein said phosphoric acid alkyl ester is represented by general formula: OP(OR¹)(OR²)(OR³),
wherein each of R¹, R², and R³ is independently selected from a C1 to C10 aliphatic group (having no aromatic ring), a C6 to C20 aryl group, a C7 to C30 alkylaryl group, and a C7 to C30 arylalkyl.

4. The chemical sensor device according to claim 1, wherein said sensitive film includes grapheme.

5. The chemical sensor device according to claim 1, further comprising:
a liquid supply mechanism that supplies a specimen liquid to a surface of said sensitive film; and
a liquid discharge mechanism that discharges said specimen liquid on said surface of said sensitive film.

6. The chemical sensor device according to claim 1, further comprising:
a source electrode that is connected to said sensitive film; and
a drain electrode that is connected to said sensitive film.

7. The chemical sensor device according to claim 1, wherein said nucleic acid compound has an immobilized site suitable for binding to said sensitive film and a detection site suitable for binding to a target substance.

8. The chemical sensor device according to claim 1, wherein said nucleic acid compound is obtained by modifying a nucleic acid with a polycyclic aromatic group.

9. The chemical sensor device according to claim 8, wherein said polycyclic aromatic group is a pyrenyl group.

10. The chemical sensor device according to claim 1, wherein said nucleic acid compound is obtained by modifying a nucleic acid with a labeled molecule.

11. The chemical sensor device according to claim 1, wherein said nucleic acid compound is immobilized on said sensitive film by a n-n interaction.

12. The chemical sensor device according to claim 1, detecting a field effect transistor signal.

13. A method for detecting a target substance, said method comprising, in the stated order:
exposing a sensitive film to a first solution containing a phosphoric acid derivative;
exposing said sensitive film to a second solution containing a nucleic acid compound; and
exposing said sensitive film to a specimen liquid capable of containing a target substance.
